(19) United States
(12) Patent Application Publication
(10) Pub. No.: US 2013/0144220 A1
(43) Pub. Date: Jun. 6, 2013

(54) SYRINGE FLANGE PROTECTOR

(71) Applicants: Ian Cleathero, Leicestershire (GB); Neil Bentley Cammish, Greater Manchester (GB)

(72) Inventors: Ian Cleathero, Leicestershire (GB); Neil Bentley Cammish, Greater Manchester (GB)

(21) Appl. No.: 13/513,652

(22) PCT Filed: Dec. 6, 2010
(86) PCT No.: PCT/GB2010/052029

§ 371 Date: Sep. 10, 2012

(30) Foreign Application Priority Data
Dec. 7, 2009 (GB) 0921384.4

(51) Int. Cl. A61M 5/315

(52) U.S. Cl. CPC A61M 5/31501
USPC 604/218

(57) ABSTRACT

A syringe flange protector for transmitting an axial force from a drive element to a syringe barrel, the syringe barrel having a flange projecting radially from the barrel; comprising a spacer element that is adapted to be disposed axially rearward of at least part of the flange, and is further adapted to communicate with the drive element through one or more points at a first radial distance from a longitudinal axis of the spacer element, and to transmit axial force from the drive element to the barrel through one or more points at a second radial distance from the longitudinal axis of the spacer element, where the second radial distance is less than the first radial distance.

This invention relates to a syringe flange protector, and in particular to a syringe flange protector for use in association with an autoinjector device.

### BACKGROUND

Standard pre-filled syringes, such as those used in autoinjector devices, are generally made from glass. An example of an autoinjector device employing a standard pre-filled syringe is described in WO-A-2007/083115 (The Medical House plc). In that autoinjector device, a drive element acts on the flange of the syringe to advance it axially forwards to insert the needle into an injection site. The load is applied axially by the drive element, which causes a bending moment in the flange. The flange then transmits the force to axially load the syringe barrel causing it to advance forwards.

A known problem associated with some standard pre-filled syringes is that the flanges are often routinely out of specification (e.g. where their actual thickness is less than their specified thickness). Coupled with the inherent brittleness of glass, this fact means that there is an associated risk of breaking the flange when a standard pre-filled syringe is used in an autoinjector device.

The present invention seeks to overcome this problem to allow for safer and more reliable autoinjector devices.

### BRIEF SUMMARY OF THE DISCLOSURE

In accordance with a first aspect of the present invention there is provided a syringe flange protector for transmitting an axial force from a drive element to a syringe barrel, the syringe barrel having a flange projecting radially from the barrel;
comprising a spacer element that is adapted to be disposed axially rearward of at least part of the flange, and is further adapted to communicate with the drive element through one or more points at a first radial distance from a longitudinal axis of the spacer element, and to transmit axial force from the drive element to the barrel through one or more points at a second radial distance from the longitudinal axis of the spacer element, where the second radial distance is less than the first radial distance.

Therefore, the syringe flange protector can provide a means for transmitting the axial force from the drive element to the syringe barrel without exerting a substantial force on the flange, thereby reducing the risk of accidental breakage of the flange.

Preferably, the spacer element is adapted to be disposed in an axially spaced relationship with substantially all of the flange. In this preferable embodiment, substantially no axial force is transmitted to the flange in use, thereby effectively mitigating the risk of accidental breakage of the flange entirely.

In a further preferable embodiment, the spacer element comprises an axially extending inner spigot for insertion in the barrel, an intermediate portion for transmitting the axial force to the barrel through the one or more points at said second radial distance, and an outer rim for communicating with the drive element through the one or more points at said first distance. The inner spigot locates the spacer element on the syringe and prevents it from moving during use, whilst the intermediate portion and the outer rim act to transmit the force from the drive element to the barrel. In a particularly preferable embodiment, the spacer element is tapered between the intermediate portion and the outer rim. Further preferably, the second radial distance is equal to the radius of the barrel. In this embodiment, the force is transmitted to the syringe barrel along a single axis and reduces any torque which may lead to breakage.

In one preferable embodiment, the spacer element is flexible. In this embodiment, the spacer element flexes in response to the axial load received from the drive element. The spacer element therefore acts like a spring and reduces the impact of the drive element on the syringe. Thus, the load is applied to the syringe over a longer time period, and the peak force is consequently reduced. This feature further serves to reduce the risk of accidental breakage of the syringe.

Preferably, the spacer element is adapted to be secured to securing means of a syringe holder. Further preferably, the securing means comprises hooks extending axially from the syringe holder. Whilst the securing means do not affect the function of the spacer element, they limit its possible axial movement and ensure its location around the syringe flange.

In another embodiment, the syringe flange protector further comprises axially extending hooks that extend from the spacer element and secure the spacer element to the flange. This also serves to locate the spacer element around the syringe flange without affecting its function.

In one particularly preferable embodiment of the invention, the spacer element is integrally formed with a syringe holder for holding a syringe. In particular, the spacer element is preferably integrally connected to the syringe holder via a plurality of axially extending legs. Alternatively or additionally, the spacer element is formed in two or more parts that may flex radially apart so as to allow installation of the syringe in the syringe holder during assembly. In a particularly preferable embodiment, each of the plurality of axially extending legs is flexible thereby permitting the two or more parts to flax radially apart.

In any aspect of the present invention, the spacer element preferably comprises one or more cut-outs for communicating with the drive element through the one or more points at the first radial distance from the longitudinal axis of the spacer element; wherein the one or more cut-outs each comprise a flat base that has a plane that is substantially perpendicular to the longitudinal axis of the spacer element, and side walls that extend substantially parallel to the longitudinal axis of the spacer element.

In accordance with a second aspect of the present invention there is provided a syringe comprising a barrel for holding a volume of medicament, a plunger axially moveable within the barrel, and a flange projecting radially from the barrel, further comprising a syringe flange protector according to the first aspect of the invention.

In accordance with a third aspect of the present invention there is provided an autoinjector device comprising a syringe according to the second aspect of the invention.

Preferably, the drive element comprises an inner housing intermediate an outer housing of the autoinjector device and the barrel and the plunger; and the inner housing is moveable by an energy source between three positions, namely:
a first position in which the inner housing is in communication with the spacer element such that, in use, the plunger and barrel are moveable axially so as to move at least part of the needle out of the outer housing;
a second position in which the inner housing is in communication with the plunger but not the spacer element such that, in use, said plunger is moveable axially into the barrel so as to expel medicament through the needle; and
a third position in which the inner housing is in communication with neither the plunger nor the barrel such that, in use, the plunger and barrel are able to retract in order to retract the needle into the outer housing.

Therefore, the syringe flange protector can provide adequate protection for the flange from the inner housing thereby reducing the risk of accidental breakage.

### BRIEF DESCRIPTION OF THE DRAWINGS

Embodiments of the invention are further described hereinafter with reference to the accompanying drawings, in which:

FIG. 1 is a perspective view of a syringe in a syringe holder including a syringe flange protector according to the present invention;

FIGS. 2A and 2B are perspective views of the top and bottom of one embodiment of a syringe flange protector according to the present invention;

FIGS. 3A and 3B are perspective views of the top and bottom of an alternative embodiment of a syringe flange protector according to the present invention;

FIG. 4 is a perspective view of a the syringe flange protector of FIGS. 3A and 3B, installed on a syringe in a syringe holder, with the axial forces exerted by a drive element indicated;

FIG. 5 is a cross-sectional view corresponding to FIG. 4, with the syringe holder removed for clarity

FIG. 6 is a perspective view of an alternative embodiment of a syringe flange protector according to the present invention, where the syringe flange protector is integrated with the syringe holder; and

FIG. 7 is a side view of the syringe flange protector of FIG. 6.

### DETAILED DESCRIPTION

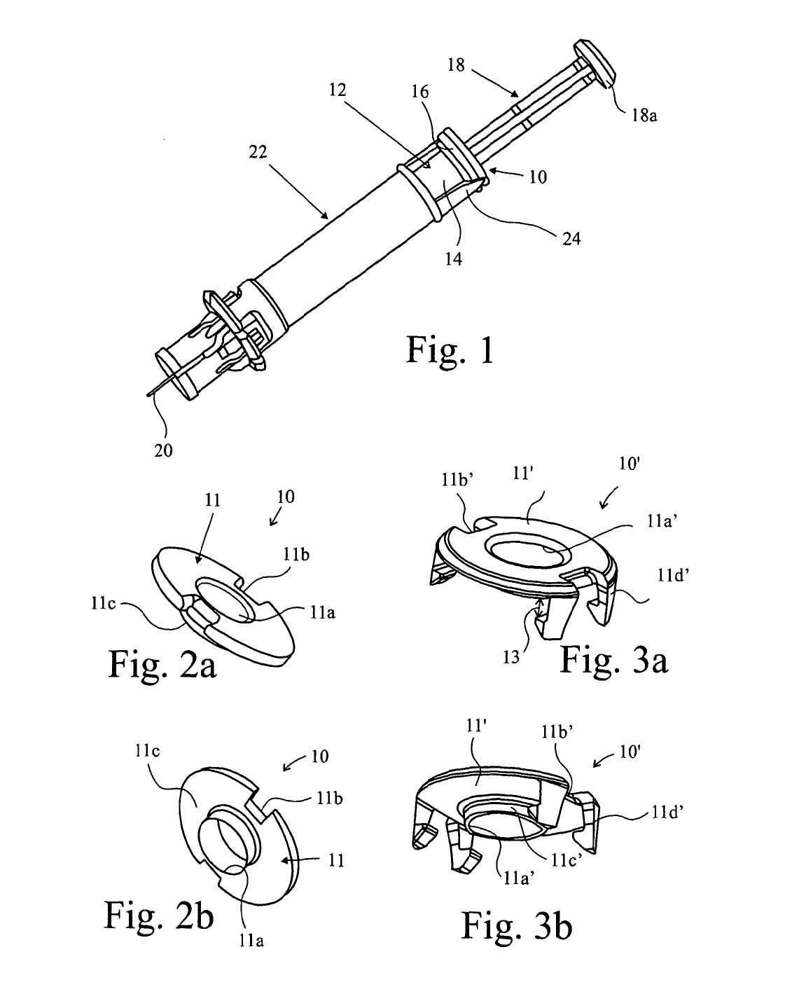

FIG. 1 shows a standard pre-filled syringe 12 held in a syringe holder 22 to which is attached a syringe flange protector 10. The syringe 12 comprises a barrel 14 for holding a volume of medicament, a plunger 18 axially moveable within the barrel 14, a needle 20 at one end of the barrel 14, and a flange 16 projecting radially from the barrel 14 at an end remote from the needle 20. The syringe 12 is supported in the syringe holder 22 which is a typical component found in autoinjector devices. In a preferable embodiment, the syringe holder 22 is equivalent to the syringe support means described in WO-A-2007/083115. The syringe holder 22 has hooks 24 that extend axially rearwardly (i.e. away from the needle 20 end) and which latch on to the syringe flange protector 10 to maintain it in a protecting position axially close to the syringe flange 16.

One embodiment of the syringe flange protector 10 is shown in FIGS. 2A and 2B. Referring to FIG. 2A, the syringe flange protector 10 comprises a spacer element 11 that has a central aperture 11a. During assembly, the syringe flange protector 10 is assembled onto the syringe 12 by passing the plunger 18 through the central aperture 11a so that the syringe flange protector 10 is radially restrained on the syringe 12. As shown more clearly in FIG. 2B, the syringe flange protector 10 has an axially extending inner spigot 11c that projects from the edges of aperture 11a. The syringe flange protector 10 also has ledges 11b that are shaped to receive the ends of the hooks 24 of the syringe holder 22. Therefore, when installed on the syringe 12, the syringe flange protector 10 can be axially restrained by the hooks 24 in relation to the syringe 12 and syringe holder 22, rearward of the flange 16. The spigot 11c slots into the barrel 14 around the periphery of the plunger 18 and limits radial displacement of the syringe flange protector 10 relative to the syringe 12. The spigot 11c may form a friction fit with the barrel 14 so that the syringe flange protector 10 is also axially restrained with respect to the syringe 12 by virtue of the friction.

A second embodiment of the syringe flange protector 10′ is shown in FIGS. 3A and 3B. The second embodiment 10′ differs from the first embodiment 10 in that it further comprises axially extending hooks 11d′ that project from the spacer element 11′ in the same direction as the spigot 11c′. The hooks 11d′ can be used to clip the syringe flange protector 10′ to the flange 16 of the syringe 12 thereby limiting the amount of possible relative axial movement therebetween. The hooks 11d′ are shaped to latch under the front side of the flange 16 so that some relative axial movement along direction 13 may be permitted.

As shown more clearly in FIG. 5, the underside (i.e. the side from which the spigot 11c,11c′ projects) of the spacer element 11, 11′ is tapered in a radial direction away from the spigot 11c,11c′. In an alternative embodiment, the spacer element 11,11′ may have a stepped profile instead of a tapered profile.

In some embodiments, the syringe flange protector 10 may be made of a resilient plastics material, however, other materials are envisaged in alternative embodiments.

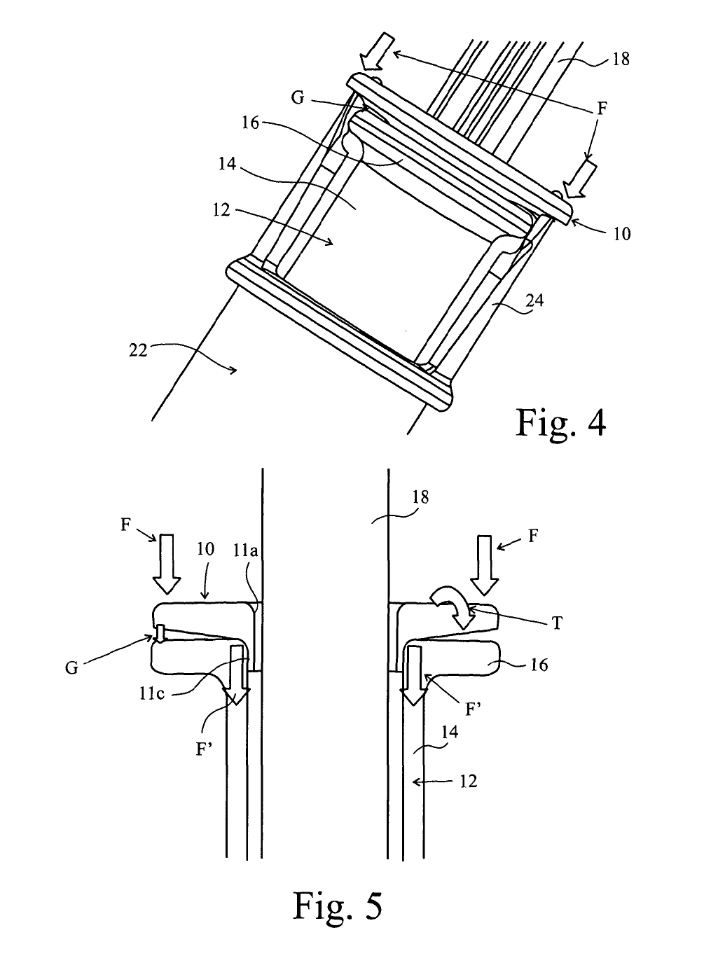

FIGS. 4 and 5 show the first embodiment of the syringe flange protector 10 assembled on the syringe 12. The skilled reader will appreciate that the second embodiment 10′, and indeed further alternative embodiments of the syringe flange protector 10, may be assembled in the same manner and fundamentally operate in the same way.

In autoinjector devices, such as the ones described in WO-A-2007/083115 and EP-B-1715903, a drive element acts on the syringe to move the needle out of the housing of the device to penetrate an injection site, and to inject medicament. In some prior art devices, the drive element acts directly on the fragile glass flange of the syringe to move the needle out of the housing. In the present invention, the syringe flange protector 10 protects the flange 16 by transmitting the axial force from the drive element to the barrel 14 of the syringe 12 without allowing the flange 16 to be stressed unduly. In some embodiments, a part of the flange 16 may still transmit a proportion of the axial load from the drive element, but this will occur radially inwards from the outer rim of the flange 16 so that the risk of accidental breakage is still reduced. In an exemplary embodiment, the syringe flange protector 10 transmits the axial load from the drive element to the barrel 14 without stressing the flange 16 at all.

In FIGS. 4 and 5, a gap G can be seen to be present between the syringe flange protector 10 and the flange 16, due to the tapered profile of spacer element 11. When the drive element (not shown) advances forward to move the needle 20 out of the housing (not shown) of an autoinjector device (not shown), the axial force F is transmitted to the syringe flange protector 10. In many cases, the drive element will be a tubular element that moves axially whilst being radially larger than the plunger 18 and a head 18a of the plunger (see FIG. 1). In many cases, therefore, the force F will be transmitted to the syringe flange protector 10 at one or more radially outward points. The load F experienced by the syringe flange protector 10 causes torque T within the spacer element 11, which consequently leads to axial loading of the syringe barrel with a force F′. In the exemplary embodiment shown in FIG. 5, the torque T is transmitted to the syringe 12 as force F′ at points that are axially aligned with the walls of the barrel 14. Therefore, substantially no load is transmitted to the flange 16 and the risk of accidental breakage is significantly reduced.

In the case where the syringe flange protector 10 is made of a resilient material, the spacer element 11 may deform under the axial loading F so that the gap G is reduced in size or even closed. However, the deformability of the spacer element 11 can be designed so that it does deform, but not to the extent where any significant loading is exerted on the flange 16.

The deformability of the spacer element 11 also serves to reduce the impact of the drive element on the syringe 12 and ensures that the load F′ is applied to the syringe 12 over a longer period, thereby reducing the peak force experienced by any part of the syringe 12 and reducing the risk of accidental breakage. The spacer element 11 therefore provides a “cushioning effect”, cushioning the syringe 12 from the force of the drive element.

Although the syringe flange protector 10 can be used to protect the flange 16 from the force otherwise directly exerted by any drive element in an autoinjector device, it is particularly suited to protecting a flange from the inner housing component of the autoinjectors described in WO-A-2007/083115 and EP-B-1715903.

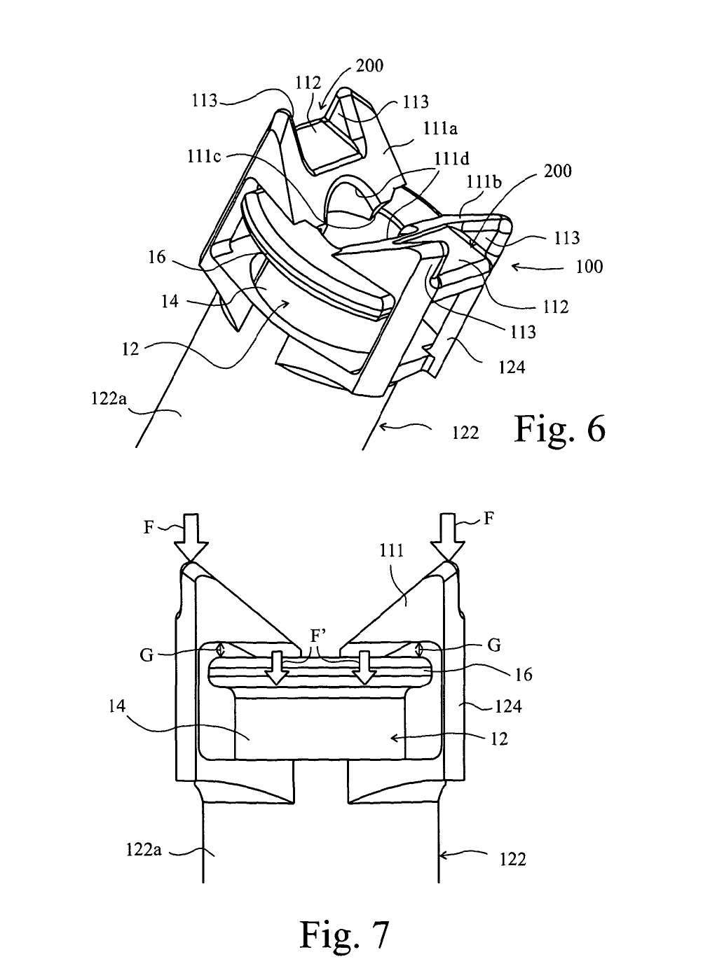

An alternative embodiment of the present invention is shown in FIGS. 6 and 7. In this alternative embodiment, a syringe flange protector 100 is integrally formed with a modified syringe holder 122. The modified syringe holder 122 is still capable of holding a standard syringe 12 that comprises a barrel 14 for holding a volume of medicament, a plunger (not shown) axially moveable within the barrel 14, a needle (not shown) at one end of the barrel 14, and a flange 16 projecting radially from the barrel 14 at an end remote from the needle 20. Like the syringe holder 22 of FIGS. 1 and 4, the syringe holder 122 of FIGS. 6 and 7 is capable of supporting the syringe 12 therein within an autoinjector device. As with the syringe holder 22 of FIGS. 1 and 4, the syringe holder 122 of FIGS. 6 and 7 is, in a preferable embodiment, equivalent to the syringe support means described in WO-A-2007/083115.

The syringe flange protector 100 is integrally connected to a main body 122a of the syringe holder 122 by four axially extending legs 124. More specifically, the syringe flange protector 100 is formed of two spacer element parts 111a,111b (which collectively form a spacer element 111) that are each connected to the main body 122a by two of the four axially extending legs 124. The four axially extending legs 124 are flexible so as to allow the two parts 111a,111b of the spacer element 111 to flex radially and allow a syringe 12 to be installed in the syringe holder 122 during assembly.

The two parts 111a,111b of the spacer element 111 are effectively equivalent to two segments of the spacer element 11 shown in FIG. 1. Similarly, the two parts 111a,111b of the spacer element 111 are shaped to define a central aperture 111d when the axially extending legs 124 are in their relaxed positions. About the central aperture 111d, the syringe flange protector 100 has an axially extending inner spigot 111c that is also formed in two parts, where each part extends axially from one of the two spacer element parts 111a,111b. When a syringe 12 is installed in the modified syringe holder 112, the axially extending inner spigot 111c extends into a bore of the syringe flange 16 slightly, but still permits a plunger (not shown) to pass therethrough and move axially within the barrel 14 to discharge medicament through the needle (not shown). Around this bore in the syringe flange 16, the spacer element 111 contacts the syringe flange 16. Radially outwards of this position, there exists a gap G between the spacer element and the syringe flange 16 (see FIG. 7). Unlike the spacer element 11 of FIG. 1, the spacer element 111 of FIGS. 6 and 7 is not particularly flexible (i.e. in axial directions). In alternative embodiments, however, the spacer element 111 may indeed be axially flexible.

At a position radially outward of the spigot 111c, each part 111a,111b of the spacer element 111 has a cut-out portion 200 that is defined by a flat base 112 and side walls 113. The plane of the flat base 112 is substantially perpendicular to a central longitudinal axis of the spacer element 111 and the side walls 113 extend axially rearward from the spacer element 111 (i.e. substantially parallel to the longitudinal axis). The flat bases 112 have a large surface area and are configured to be the points of contact between the syringe flange protector 100 and the drive element (not shown). The side walls 113 are configured to define the areas where the drive element makes contact with the spacer element 111 and ensure that this connection is secure during motion.

Due to the large surface area of the flat bases 112, a force F received from the drive element does not cause considerable pressure on the spacer element 111 (due to the relationship: pressure=force/area) and so the risk of breakage or damage of the spacer element 111 is reduced. Furthermore, the surface area of the points of contact between the spacer element 111 and the syringe 12 is preferably large, also, to reduce the pressure transmitted to the syringe 12 due to the force F from the drive element through the spacer element 111, and thereby minimize the risk of breakage of the syringe 12. The principles of using large surface areas on the spacer element to receive and/or transmit forces from the drive element/to the syringe 12 may be employed in any embodiment according to the present invention. Indeed, the cut-outs described in connection with the spacer element 111 of FIGS. 6 and 7 may be employed in other embodiments of the present invention.

Returning specifically to the embodiment shown in FIGS. 6 and 7, the points of contact between the spacer element 111 and the syringe 12 are preferably axially aligned with the walls of the barrel so that the force F′ transmitted from the spacer element 111 to the syringe 12 does not pass through the syringe flange 16 thereby protecting the syringe flange 16 from any potential damage.

In alternative embodiments, the spacer element 111 may comprise any number of segmented pieces provided that they are configured to permit installation of the syringe 12 in the syringe holder 122. In embodiments where the spacer element 111 is integral with the syringe holder 122, the segmented pieces of the syringe holder 111 may be connected to the main body of the syringe holder 122 by elements other than axially extending legs, although a flexible component may be preferable so that installation of the syringe 12 is facilitated. In embodiments where axially extending legs are present, any number of flexible legs may be present and the present invention is not limited to having two legs per segmented piece of the spacer element.

Fundamentally, the syringe flange protector 10,10′,100 transmits the axial load of a drive element by communicating with the drive element through one or more points at a first radial distance, and transmitting the axial force from the drive element to the barrel through one or more points at a second radial distance, where the second radial distance is less than the first radial distance. It is to be understood that the term “radial distance” means the radial distance from a central longitudinal axis of the spacer element 11,11′,111. Indeed, when the syringe flange protector 10,10′,100 is coaxial with the syringe 12 (i.e. when the syringe flange protector 10,10′,100 is assembled on the syringe 12), “radial distance” may also be taken to mean the radial distance from a central longitudinal axis of the syringe 12.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A syringe flange protector for transmitting an axial force from a drive element to a syringe barrel, the syringe barrel having a flange projecting radially from the barrel;
comprising a spacer element that is adapted to be disposed axially rearward of at least part of the flange, and is further adapted to communicate with the drive element through one or more points at a first radial distance from a longitudinal axis of the spacer element, and to transmit axial force from the drive element to the barrel through one or more points at a second radial distance from the longitudinal axis of the spacer element, where the second radial distance is less than the first radial distance.

2. A syringe flange protector according to claim 1, wherein the spacer element is adapted to be disposed in an axially spaced relationship with substantially all of the flange.

3. A syringe flange protector according to claim 1, wherein the spacer element comprises an axially extending inner spigot for insertion in the barrel, an intermediate portion for transmitting the axial force to the barrel through the one or more points at said second radial distance, and an outer rim for communicating with the drive element through the one or more points at said first distance.

4. A syringe flange protector according to claim 3, wherein the spacer element is tapered between the intermediate portion and the outer rim.

5. A syringe flange protector according to claim 3, wherein the second radial distance is equal to the radius of the barrel.

6. A syringe flange protector according to claim 1, wherein the spacer element is flexible.

7. A syringe flange protector according to claim 1, wherein the spacer element is adapted to be secured to securing means of a syringe holder.

8. A syringe flange protector according to claim 7, wherein the securing means comprises hooks extending axially from the syringe holder.

9. A syringe flange protector according to claim 1, further comprising axially extending hooks that extend from the spacer element and secure the spacer element to the flange.

10. A syringe flange protector according to claim 1, wherein the spacer element is integrally formed with a syringe holder for holding a syringe.

11. A syringe flange protector according to claim 10, wherein the spacer element is integrally connected to the syringe holder via a plurality of axially extending legs.

12. A syringe flange protector according to claim 11, wherein the spacer element is formed in two or more parts that may flex radially apart so as to allow installation of the syringe in the syringe holder during assembly.

13. A syringe flange protector according to claim 12, wherein each of the plurality of axially extending legs is flexible thereby permitting the two or more parts to flax radially apart.

14. A syringe flange protector according to claim 1, wherein the spacer element comprises one or more cut-outs for communicating with the drive element through the one or more points at the first radial distance from the longitudinal axis of the spacer element; wherein the one or more cut-outs each comprise a flat base that has a plane that is substantially perpendicular to the longitudinal axis of the spacer element, and side walls that extend substantially parallel to the longitudinal axis of the spacer element.

15. (canceled)

16. A syringe comprising a barrel for holding a volume of medicament, a plunger axially moveable within the barrel, and a flange projecting radially from the barrel, further comprising the syringe flange protector of claim 1.

17. An autoinjector device comprising the syringe of claim 16.

18. An autoinjector device according to claim 17, wherein the drive element comprises an inner housing intermediate an outer housing of the autoinjector device and the barrel and the plunger; and the inner housing is moveable by an energy source between three positions, namely:
a first position in which the inner housing is in communication with the spacer element such that, in use, the plunger and barrel are moveable axially so as to move at least part of the needle our of the outer housing;
a second position in which the inner housing is in communication with the plunger but not the spacer element such that, in use, said plunger is moveable axially into the barrel so as to expel medicament through the needle; and
a third position in which the inner housing is in communication with neither the plunger nor the barrel such that, in use, the plunger and barrel are able to retract in order to retract the needle into the outer housing.

19. (canceled)

20. A syringe flange protector according to claim 12, wherein the spacer element comprises one or more cut-outs for communicating with the drive element through the one or more points at the first radial distance from the longitudinal axis of the spacer element;
wherein the one or more cut-outs each comprise a flat base that has a plane that is substantially perpendicular to the longitudinal axis of the spacer element, and side walls that extend substantially parallel to the longitudinal axis of the spacer element.

21. A syringe flange protector according to claim 20, wherein the spacer element is formed in two parts and each part comprises a cut-out.
